(19) United States
(12) Patent Application Publication
(10) Pub. No.: US 2005/0261682 A1
(43) Pub. Date: Nov. 24, 2005

(54) Vertebral shock absorbers

(76) Inventor: Bret A. Ferree, Cincinnati, OH (US)

Correspondence Address: John G. Posa;Gifford, Krass, Groh, Sprinkle,, Anderson & Citkowski, P.C., 280 N. Old Woodward Ave., Suite 400, Birmingham, MI, 48009-5394, US

(21) Appl. No.: 10/412,896

(22) Filed: Apr. 14, 2003

Related U.S. Application Data
(60) Provisional application No. 60/372,754, filed on Apr. 13, 2002.

(51) Int. Cl. A61B 17/70

(52) U.S. Cl. 606/61

(57) ABSTRACT

A vertebral shock absorber in the form of an elongated compressible member having two ends, one fastened to an upper vertebra, and the other fastened to a lower vertebra. The elongated member may be fastened using pedicle screws or by way of ball-and-socket joints for enhanced range of motion. In the preferred embodiment, the elongated compressible member is constructed using telescoping sleeves to create a cavity wherein there is disposed a compressible, resilient component such as a spring, elastomeric material, liquid, gel, hydrogel, or other suitable substance. The shock absorber may be combined with an intradiscal component and/or one or more plates that extends at least partially onto a vertebral endplate to facilitate lateral application.

### REFERENCE TO RELATED APPLICATION

This application claims priority from U.S. Provisional Patent Application Ser. No. 60/372,754, filed Apr. 13, 2002, the entire content of which is incorporated herein by reference.

### FIELD OF THE INVENTION

This invention relates generally to spinal surgery and, in particular, to shock-absorbing structures spanning vertebral levels.

### BACKGROUND OF THE INVENTION

The problems created by disc degeneration, facet arthritis, and other conditions such as spondylolysis, spondylolisthesis, scoliosis, fracture, tumor, or infection are frequently treated by spinal fusion. Such problems may include pain in the back or legs, nerve injury, risk of future nerve injury, or spinal deformity.

The goal of spinal fusion is to successfully “grow” two or more vertebrae together. To achieve this, bone from the patient's body (spine or iliac crest) or from cadavers, is often grafted between vertebrae. Alternatively, bone graft substitutes, such as hydroxy appetite and bone morphogenetic protein, may be used. The bone graft is placed between the vertebrae in the disc space and/or over the posterior elements of the vertebrae (lamina and transverse processes).

Other instrumentation, including cages, is placed onto or into the spine to immobilize the vertebrae that are going to be fused. Cages immobilize the vertebrae and maintain the separation between the vertebrae, a function of the former disc material. Cages may be combined with rods, screws, hooks, or plates. Combining cages with other instrumentation yields a stiffer construct and presumably increases the chance of a successful fusion. If cages are placed from an anterior approach (through the abdomen) the patient must undergo surgery through a second incision over the back (posterior approach) if the surgeon wants to also insert rods and screws. To avoid two incisions and increased patient morbidity, many surgeons prefer to insert the cages from a posterior approach. Rods and screws can be added through the same incision.

Pedicle screws are generally connected by solid rods or plates in an attempt to eliminate spinal motion. A few inventors have connected pedicle screws with rubber, elastic, or fibrous materials to dampen or restrict spinal motion. These inventors have postulated that low back pain is caused by abnormal movements and/or pressure across the facet joints.

U.S. Pat. No. 5,415,661 discloses the use of pedicle screws connected by fibrous bands to limit flexion of the spine (distraction of the posterior portion of the vertebrae). The devices were improved by covering the fibrous bands with rubber sleeves. The rubber sleeves help dampen the forces on the facets that occurs with spinal extension. That is, the rubber sleeves help prevent extension of the spine. Forces on the facets increase with extension. Alternative shock-absorbing devices are disclosed in U.S. Pat. Nos. 4,932,975; 5,480,401; 5,540,688; and 5,961,516.

Although such devices provide some level of shock absorption, the use of a fibrous band or cushioning around a small joint does not make efficient use of components of materials for this purpose, and could lead to premature wear or failure. Thus, the need remains for an improved shock absorber that is structurally and functionally superior in terms of performance and longevity.

### SUMMARY OF THE INVENTION

This invention improves upon existing devices by providing a vertebral shock absorber in the form of an elongated compressible member having two ends, one fastened to an upper vertebra, and the other fastened to a lower vertebra. The elongated member may be fastened using pedicle screws or by way of ball-and-socket joints for enhanced range of motion.

In the preferred embodiment the elongated compressible member is constructed using telescoping sleeves to create a cavity wherein there is disposed a compressible, resilient component such as a spring, elastomeric material, liquid, gel, hydrogel, or other suitable substance. The shock absorber may be combined with an intradiscal component and/or one or more plates that extends at least partially onto a vertebral endplate to facilitate lateral application.

### BRIEF DESCRIPTION OF THE DRAWINGS

FIG. 1A is a drawing which illustrates a posterior application of the invention;

FIG. 1B is a lateral view of the application of FIG. 1A;

FIG. 2 is a coronal cross section of the spine and an embodiment of the invention for the anterior or lateral aspect of the spine;

FIG. 3 is a view of the lateral side of the spine and the embodiment of the invention drawn in FIG. 2

FIG. 4A is a coronal cross section of the spine and an alternative embodiment of the invention wherein plates on the lateral side of the spine do not extend into the disc space;

FIG. 4B is a view of the front of a lateral flexed spine and a coronal cross section of the embodiment of the invention drawn in FIG. 4A;

FIG. 4C is a view of the lateral side of a flexed spine and the embodiment of the invention drawn in FIG. 4A;

FIG. 5 is a coronal cross section of a vertebral shock absorber showing a spring and a fluid-filled damper component; and

FIG. 6 is a view of the lateral surface of the spine and an embodiment of the invention with two shock absorbers on the lateral side of the spine.

### DETAILED DESCRIPTION OF THE INVENTION

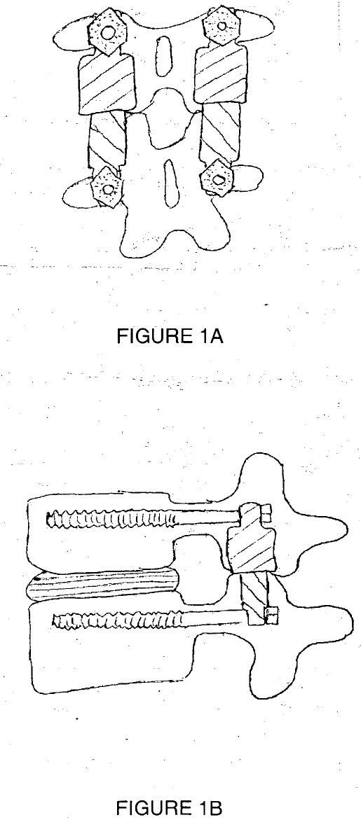

Broadly, this invention covers the use of “shock absorbers” on the posterior, lateral, or anterior aspect of the spine. FIG. 1A is a view of the posterior aspect of the posterior embodiment of the invention and the spine. FIG. 1B is sagittal cross section of the spine and the embodiment of the device drawn in FIG. 1A. In the posterior embodiment, the vertebral shock absorbers are connected to pedicle screws. Note that the devices could be used as an adjunct to arthroplasty devices placed in the disc space.

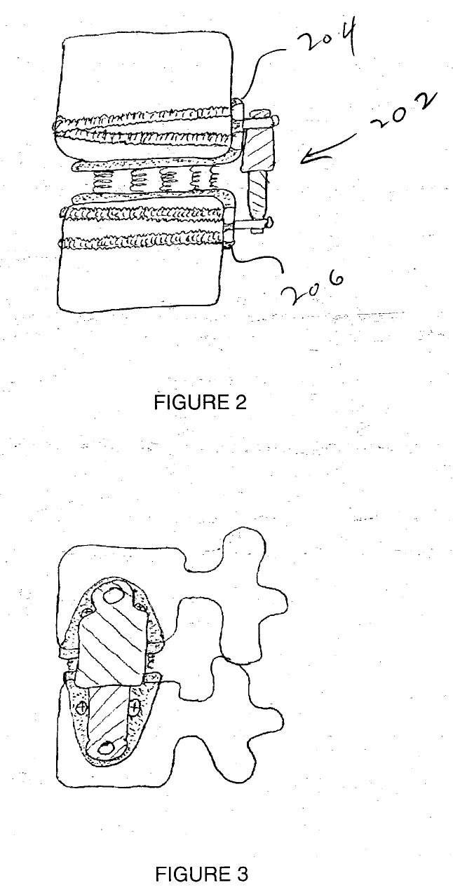

FIG. 2 is a coronal cross section of the spine and an embodiment of the invention particularly suited to the anterior or lateral aspect of the spine. FIG. 3 is a view of the lateral side of the spine and the embodiment of the invention drawn in FIG. 2. In this case a vertebral shock absorber 202 is connected to plates 204, 206 that are attached to the lateral aspect of the spine. The plates are preferably connected to the spine by diverging or converging screws. The screws may be treaded into the plate to affix the screws to the plate in a rigid fashion. Alternatively, the plate may incorporate features, similar to those used in plates for the cervical spine, that prevent the screws from backing out of the plates.

One or both plates may further include a component that extends into the disc space. For example, the plate may have a component that extends over the vertebral endplates, as shown in the drawing. Springs, elastomeric components, hydrogel components, or artificial disc replacements could be placed between the intradiscal arms of the plates.

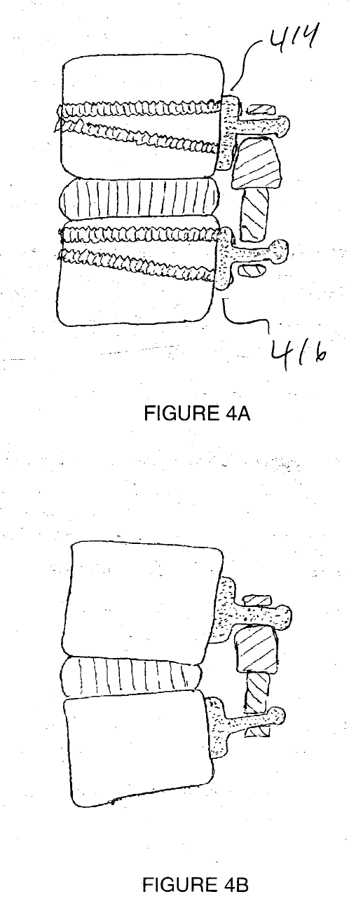

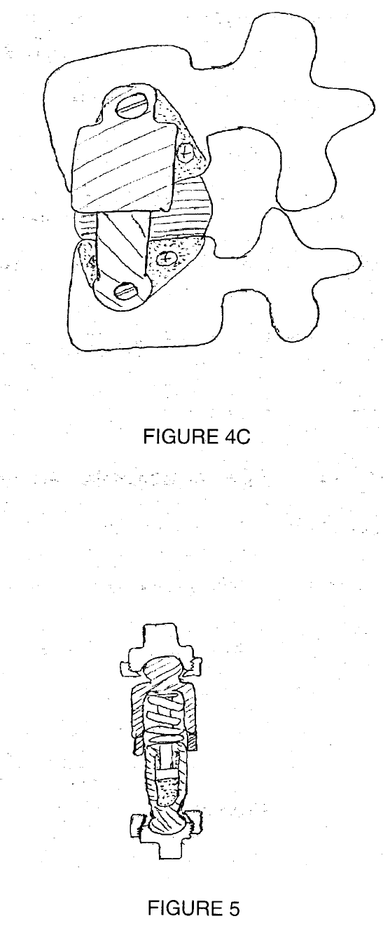

FIG. 4A is a coronal cross section of the spine and an alternative embodiment of the invention including plates 414, 416 on the lateral side of the spine that do not extend into the disc space. FIG. 4B is a view of the front of a lateral flexed spine and a coronal cross section of the embodiment of the invention drawn in FIG. 4A. FIG. 4C is a view of the lateral side of a flexed spine and the embodiment of the invention drawn in FIG. 4A. A loose connection is maintained between the shock absorber and the attachment component allows lateral bending of the spine. The shock absorber rotates about the attachment component to allow spinal flexion and spinal extension.

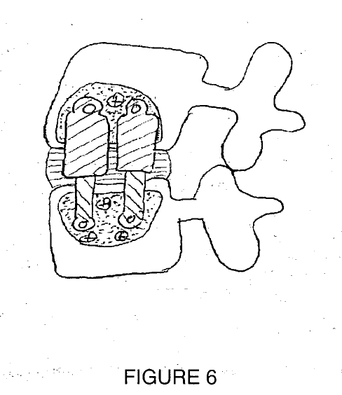

FIG. 5 is a coronal cross section of a vertebral shock absorber and an alternative attachment mechanism. The cross section of the shock absorber shows a spring and a fluid-filled damper component. The dotted area of the drawing represents the fluid. This shock absorber design may also be connected to the attachment plate through spherical joints. The sphere on the end of the shock absorber is contained in concavities of the attachment plate. A nut loosely holds the sphere within the concavity. Spherical joints allow 360 degrees of motion.

FIG. 6 is a view of the lateral surface of the spine and an embodiment of the invention with two shock absorbers on the lateral side of the spine.

1. A vertebral shock absorber, comprising:
an elongated compressible member having two ends, one fastened to an upper vertebra, and the other fastened to a lower vertebra.

2. The vertebral shock absorber of Claim 1, wherein the ends of the elongated member are fastened using pedicle screws.

3. The vertebral shock absorber of claim 1, wherein the ends of the elongated member are fastened using ball-and-socket joints.

4. The vertebral shock absorber of claim 1, wherein the elongated compressible member is constructed using telescoping sleeves.

5. The vertebral shock absorber of claim 1, wherein:
the elongated compressible member is constructed using telescoping sleeves to create a cavity therewithin; and
a compressible, resilient component disposed within the cavity.

6. The vertebral shock absorber of claim 1, wherein the compressible, resilient component is a spring.

7. The vertebral shock absorber of claim 1, wherein the compressible, resilient component is a spring.

8. The vertebral shock absorber of claim 1, wherein the compressible, resilient component is an elastomeric.

9. The vertebral shock absorber of claim 1, wherein the compressible, resilient component is a hydrogel.

10. The vertebral shock absorber of claim 1, further including an intradiscal component.

11. The vertebral shock absorber of claim 1, further including a plate that extends at least partially onto a vertebral endplate to facilitate lateral application.
